(19) United States
(12) Plant Patent Application Publication
(10) Pub. No.: US 2007/0101470 P1
(43) Pub. Date: May 3, 2007

(54) Forsythia plant named 'Courtalyn'

(75) Inventor: M. Alain Cadic, Beaucouze (FR)

Correspondence Address: BUCHANAN, INGERSOLL & ROONEY PC, POST OFFICE BOX 1404, ALEXANDRIA, VA, 22313-1404, US

(73) Assignee: AGRI OBTENTIONS S.A, Guvancourt cedex (FR)

(21) Appl. No.: 11/640,303

(22) Filed: Dec. 18, 2006

Related U.S. Application Data
(63) Continuation of application No. 11/071,176, filed on Mar. 4, 2005.
Application No. 11/071,176 is a continuation of application No. 10/217,519, filed on Aug. 14, 2002, now abandoned.
Application No. 10/217,519 is a continuation of application No. 09/322,161, filed on May 28, 1999, now abandoned.

(51) Int. Cl. A01H 5/00

(52) U.S. Cl. PLT/230

(57) ABSTRACT

A new and distinct cultivar of Forsythia plant is provided that is an induced mutation of the ‘Lynwood’ cultivar (non-patented in the United States). The gamma radiation of buds was utilized to create the mutation. The new cultivar can be readily distinguished from the parent cultivar by the presence of a more uniform and compact upright growth habit which yields a plant approximately two-thirds the height of the parent cultivar. Showy golden yellow blossoms are presented over the entire plant in the springtime prior to the appearance of the leaves. The winter hardiness is good. The new cultivar is particularly well suited for growing as attractive ornamentation in the landscape.

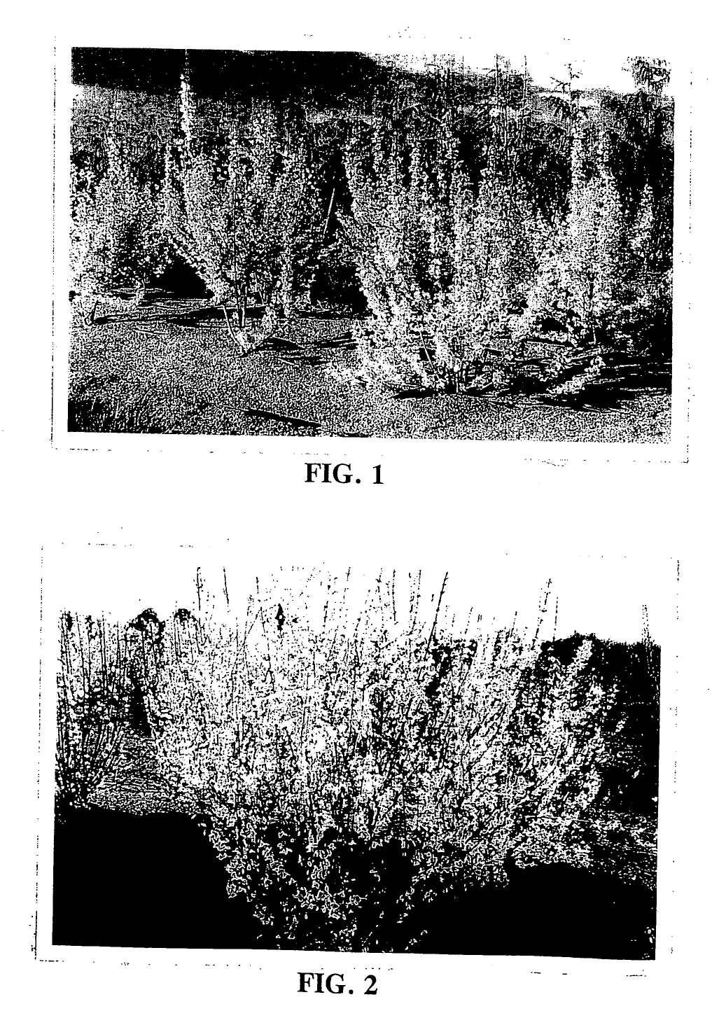

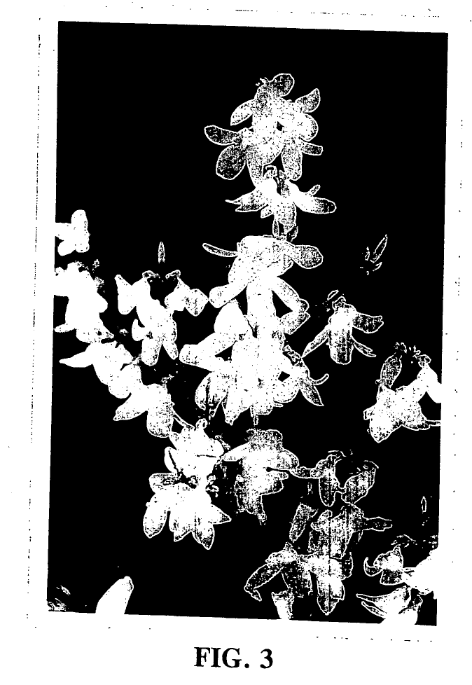

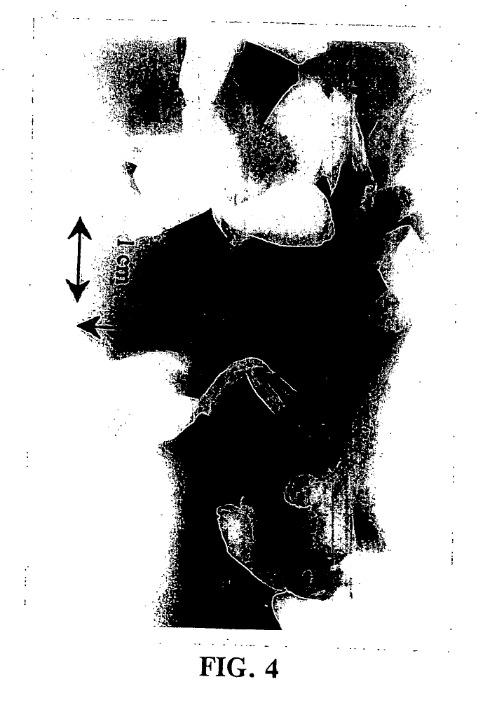

### ORIGIN

Induced mutation. Buds present on one year-old stems were irradiated with gamma rays derived from cobalt 60.

### PARENTAGE

‘Lynwood’ cultivar (non-patented in the United States).

### CLASSIFICATION

Forsythia×intermetia, cv. ‘Courtalyn’.

Plant
   Form.—well-branched flowering shrub.
   Habit.—upright, graceful, and compact. The plant has a fuller more uniform appearance with fewer wild branches than the parent ‘Lynwood’ cultivar.
   Internode length.—shorter than that of the parent ‘Lynwood’ cultivar. The internode length commonly is approximately 4.7 cm on average. This compares to an internode length for the ‘Lynwood’ cultivar of approximately 5.5 cm.
   Height.—approximately two-thirds that of the parent ‘Lynwood’ cultivar. The plant size is highly dependent upon climatic and soil conditions, as well as upon the degree of pruning (if any). A twelve year-old plant of the ‘Courtalyn’ cultivar that has been trimmed regularly commonly will exhibit a height of approximately 1.5 meters. The ‘Lynwood’ cultivar commonly will assume a height of approximately 2 meters under the same conditions.
   Width.—a twelve year-old plant of the ‘Courtalyn’ cultivar that has been trimmed regularly commonly will exhibit a width of approximately 1 meter.
   Stems.—a one year-old stem typically is Yellow-Green Group 152C in coloration. However, very young stem portions facing the sun are near Greyed-Orange Group 177A in coloration.
   Lenticels.—irregularly present between internodes in a sparse quantity, small, and commonly less than 1 mm in size.
   Branch angles.—typically approximately 30 to 40 degrees.
Foliage:
   Disposition.—opposite.
   Configuration.—substantially the same as that of the parent ‘Lynwood’ cultivar. Such configuration is lanceolate with an acuminate tip and an attenuate base.
   Leaf time.—depends upon the climatic conditions that are encountered to break dormancy. Leafing commonly occurs towards the end of March to approximately mid-April.
   Size.—generally smaller in size than those of the ‘Lynwood’ cultivar. During mid-July typical leaves from the central portion of well-developed shoots on a pruned plant commonly measure approximately 8 cm in length and approximately 2.9 cm in width. This compares to a length of approximately 10 cm and a width of approximately 3.8 cm for the ‘Lynwood’ cultivar.
   Petiole.—commonly approximately 2 cm in length. This compares to a length of approximately 2.8 cm for the ‘Lynwood’ cultivar. The coloration is near Yellow-Green Group 144B when shaded and near Yellow-Green Group 152A when fully exposed to the sun.
   Color.—substantially the same as that of the parent ‘Lynwood’ cultivar. The leaves on a fully mature limb commonly approach R.H.S. Yellow-Green Group 146A on the upper surface and Yellow-Green Group 146B on the under surface. The young growing leaves tend to be lighter and approach Yellow-Green Group 144A in coloration on the upper and under surfaces.
   Stipules.—not present.
   Leaf drop.—during mid-November at Angers, France.
Inflorescence:
   Configuration.—funnel-shaped and substantially the same as but slightly larger than the parent ‘Lynwood’ cultivar. The tube length commonly is approximately 5.8 mm. This compares to approximately 5.3 mm for the ‘Lynwood’ cultivar.
   Size.—substantially the same as the parent ‘Lynwood’ cultivar.
   Petal number.—four, and the same as the entire species.
   Petal shape.—rounded.
   Petal size.—approximately 1.6 cm in length and approximately 0.8 cm in width.
   Color.—showy, golden yellow and substantially the same as the parent ‘Lynwood’ cultivar. The same coloration is present on both petal surfaces. Commonly the coloration is Yellow Group 12A. This can be compared to Yellow Group 9A for the ‘Lynwood’ cultivar.
   Stamens.—commonly extend slightly above the pistil. The filaments commonly are approximately 3.95 mm in length and the anthers positioned on the filaments commonly measure approximately 2.45 mm. The coloration is Yellow Group 12A.
   Pistil.—commonly measures approximately 4 mm in height. The coloration is Yellow Group 12A.
   Pollen.—formed but not presently available for observation and further characterization.
   Peduncle.—approximately 10.5 mm in length, approximately 2 mm in diameter, and the coloration is light yellow-green, approaching Yellow-Green Group 154A.
   Fragrance.—none.
   Disposition.—spread substantially evenly throughout the plant (as illustrated). More freely on one year-old shoots than displayed by the ‘Lynwood’ cultivar.
   Time of flowering.—prior to formation of leaves. During 1990 to 1993 commonly bloomed for 15 to 25 days at Angers, France. The earliest blooming date was February 25th and the latest blooming date was April 10th. This compares to a blooming duration of 10 to 20 days for the parent ‘Lynwood’ cultivar. Also, the earliest blooming date was February 20th and the latest blooming date was March 30th for the ‘Lynwood’ cultivar. Often the new cultivar flowers slightly earlier than the ‘Lynwood’ cultivar.
   Lastingness.—a typical flower commonly lasts approximately 15 to 20 days on the plant. The blossom life is influenced by the weather conditions that are encountered.
   Fruit bearing.—none.
Growing conditions: Does well in full sun, and prefers soil that is not exceedingly dry.
Disease resistance: Substantially the same as that of the parent ‘Lynwood’ cultivar and not particularly susceptible to diseases. The slight presence of Pseudomonas and Botrytis may be observed in the springtime following blooming.
Hardiness: Very good as is the parent ‘Lynwood’ cultivar. Has withstood temperatures of −18 C. at Angers, France.
Propagation: Can be readily propagated while utilizing softwood cuttings.
Usage: Attractive erect ornamental spring-flowering shrub for the landscape that requires little care.

I claim:

1. A new and distinct Forsythia cultivar that is a mutation of the ‘Lynwood’ cultivar having the following combination of characteristics:
(a) forms a more uniform and regular growth habit than the parent ‘Lynwood’ cultivar,
(b) exhibits a more compact growth habit than the parent ‘Lynwood’ cultivar with shorter internode lengths and forms a plant that is approximately two-thirds the height of the ‘Lynwood’ cultivar,
(c) forms attractive golden yellow blossoms over the entire plant in the springtime,
(d) propagates well by the use of softwood cuttings,
(e) is well suited for growing in containers, and
(f) exhibits good winter hardiness;
substantially as illustrated and described.
